(19) United States
(12) Patent Application Publication
(10) Pub. No.: US 2007/0142704 A1
(43) Pub. Date: Jun. 21, 2007

(54) Image detection and storage device

(76) Inventors: Hsien-Ming Wu, Hsinchu City (TW); Shih-Chieh Lu, Hsinchu City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/603,028

(22) Filed: Nov. 22, 2006

(30) Foreign Application Priority Data
Dec. 21, 2005 (TW) 94145429

(51) Int. Cl. A61B 1/04

(52) U.S. Cl. 600/109; 600/160

(57) ABSTRACT

The present invention discloses a novel electronic detection system, which comprises a digital detection module, a digital data storage device and a power supply device. The present invention is free from the problems occurring in wired or wireless data transmission. At present, wired data transmission and wireless data transmission respectively have their own restrictions. Wired data transmission is restricted by the location and inconvenienced by the cable. Wireless data transmission is free from the inconvenience of cables, but it is likely to be interfered with by electromagnetic waves or the environment. Further, wireless data transmission is also limited by the transmission distance and the shielding effect. The present invention integrates: the detection device and the storage device inside-a single unit; therefore, the present invention can be free from the abovementioned problems. Further, data security and storage reliability is also promoted in the present invention.

### BACKGROUND OF THE INVENTION

1. Field of the Invention

The present invention relates to a device, wherein a detection device and a data storage device are integrated inside a single unit. Thus, the present invention can be used to obtain specified parameters in the locations where data transmission is restricted, such as the interior of a living body. The detection device may be an electronic sensor, such as an image sensor, a pressure sensor, a temperature sensor, or a pH sensor. Via an electronic circuit design or an IC design, the electronic signals are digitally stored.

2. Description of the Related Art

In medicine, the endoscope is widely used in inspecting tissues and obtaining the clinical images or used to perform a small-scale surgical operation. The current optical-fiber endoscope can only inspect the oesophagus, the stomach, the large intestine and the front 90 cm of the over 3 m long small intestine. Therefore, the conventional optical-fiber endoscope is unlikely to inspect most of the small intestine. Although there is an endoscope dedicated to inspect the small intestine, it needs two physicians and over 3 hours of general anaesthesia.

An Israel company-Given Imaging-developed a wireless data transmission capsule endoscope system to solve the problems of the conventional endoscopes and disclosed the technology in a US patent publication No. 5604531, wherein image data is wirelessly transmitted. Refer to FIG. 1. The casing of the conventional wireless capsule endoscope 10 comprises a transparent front cover 12 and an opaque shell 13. When a patient swallows the wireless capsule endoscope 10, the transparent front cover 12 will closely neighbors the digestive tract 11. A light-emitting diode (LED) 14 emits light; the light passes through the transparent front cover 12 and illuminates the inner wall of the digestive tract 11. The reflected light passes through the transparent front cover 12, a front imaging lens 17a and a rear imaging lens 17b and then forms an image on a charge-coupled device (CCD) 16. A CCD driver 17 drives the CCD 16 and sends the electronic signals of the images to a wireless transmitter 18 for transmission. An external antenna collects the image signals and sends the signals to a receiver; the signals are processed by circuits and then stored or presented on a display for the diagnosis of physicians. The wireless capsule endoscope is indeed effective in obtaining the clinical images of the small intestine. However, the data thereof must be wirelessly transmitted and received. Electromagnetic waves are apt to interfere with wireless transmission; thus, some data may be dropped or distorted. Further, as the patient has to wear the wireless receiver (including an antenna and a data recorder) for a long period of time (about 8 hours), he will feel uncomfortable. Therefore, the present invention integrates the detection device and the storage device inside a single device; thus, the system can be free from the transmitter and receiver, and the patient can feel more comfortable and convenient.

### SUMMARY OF THE INVENTION

The primary objective of the present invention is to provide an endoscope system, which can obtain the clinical images of the small intestines. The endoscope system is fabricated to a swallowable size and swallowed by a testee. The swallowed endoscope system will be moved forward by the peristalsis of the gastrointestinal tract. The endoscope system of the present invention obtains the images of the small intestines with the detection device thereof and stores the digital data of the images in the storage device thereof. The swallowed endoscope system will finally pass through the gastrointestinal tract and then be excreted. The stored data will be downloaded to a dedicated platform via a transmission interface and inspected by specialist physicians.

### BRIEF DESCRIPTION OF THE DRAWINGS

FIG. 1 is a diagram schematically showing a conventional capsule endoscope;

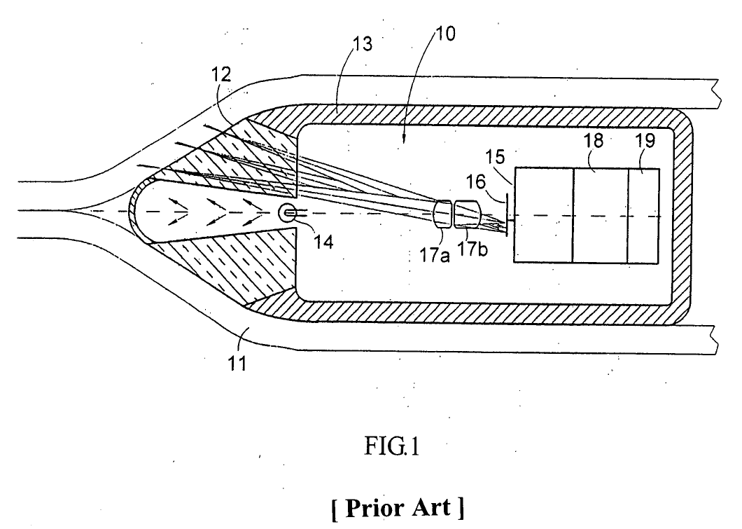

FIG. 2 is a diagram schematically showing the image detection and storage device of the present invention; and

FIG. 3 is a diagram schematically showing the function of the data processing device in the present invention.

### DETAILED DESCRIPTION OF THE INVENTION

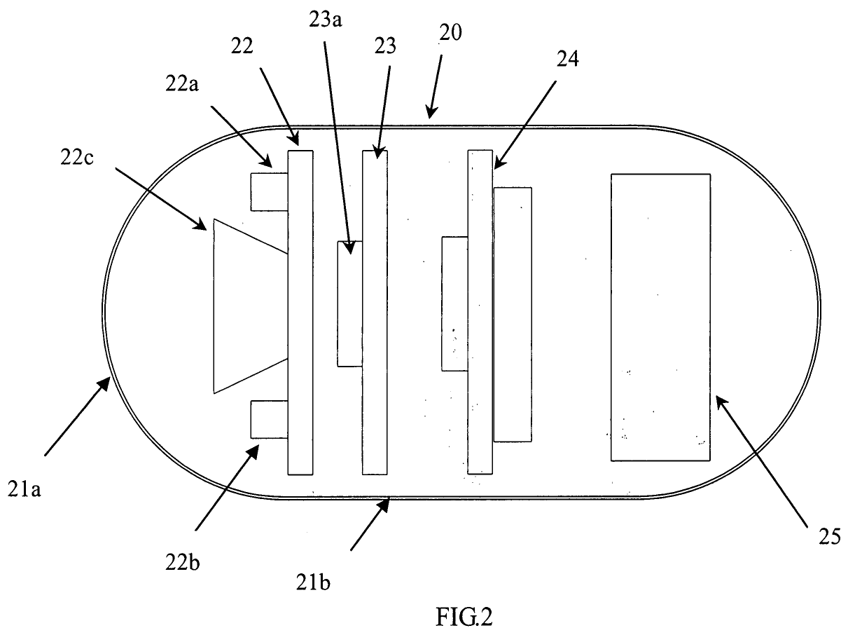

Refer to FIG. 2 a diagram schematically showing the swallowable capsular image detection and storage device 20 of the present invention. The casing of the device is made of a biologically compatible plastic and comprises a transparent front window 21a and a capsular shell 21b. The front portion of the capsular image detection and storage device 20 has a CMOS (Complementary Metal Oxide Semiconductor) image sensor 23a and an image-sensor circuit board 23. An illumination-module circuit board 22 is arranged in front of the image sensor 23a, and at least two light-emitting diodes (LED) 22a and 22b and an imaging lens module 22c are disposed on the illumination-module circuit board 22. Light is emitted from the LED's 22a and 22b and passes through the transparent front window 21a to illuminate the in vivo tissue. The light is then reflected from the illuminated tissue and passes through the transparent front window 21a again and next passes through the imaging lens module 22c and then forms an image on the CMOS image sensor 23a. The driver circuit of the CMOS image sensor 23a transforms the optical images formed on the CMOS image sensor 23a into electronic signals. The electronic signals are processed by a data processing device 24 and then stored in the memory of the data processing device 24. A power supply device 25 is installed inside the image detection and storage device 20 provides power for the LED's 22a and 22b, the CMOS image sensor the data processing device 24 and the related circuits.

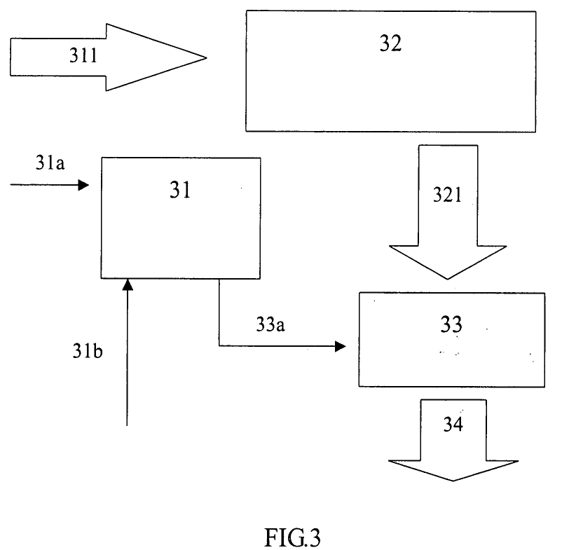

The function of the data processing device 24 is shown in FIG. 3 and can be implemented with at least one integrated circuit. When an inspection begins, a data processing unit 31 receives a data write-in instruction 31a and stores the data obtained by the sensor (a sensor data syseam 311) to a memory 32. After the inspection is completed, the system is connected to a download device, and the data processing unit 31 will receive a data read-out instruction 31b and then send a download instruction 33a to a download interface 33. Thus, the data stored in the memory 32 (a data stream 321) is transmitted to the download interface 33 and then downloaded to the exterior (34).

What is claimed is:

1. An image detection and storage device, comprising:
a casing further comprising a transparent front window and a shell;
an illumination device installed inside said transparent front window;
an optical and image-capture device installed behind said illumination device and used to transform optical signals into electronic signals;
a digital data processing device further comprising an integrated circuit and a memory; and
at least one battery connected to all said devices and providing power for said devices.

2. The image detection and storage device--according to claim 1, wherein said integrated circuit of said data processing device functions to process data, store data and download data.

3. The image detection and storage device according to claim 1, wherein said memory of said data processing device is a high capacity non-volatile memory.

4. The image detection and storage device according to claim 1, wherein said data processing device further comprises a transmission interface for data download, and said transmission interface downloads data in a serial transmission method.
